(19) United States
(12) Patent Application Publication
(10) Pub. No.: US 2016/0220606 A1
(43) Pub. Date: Aug. 4, 2016

(54) SILVER-COPPER-ZINC OXIDE WOUND CARE SYSTEM

(71) Applicant: MONIF M. MATOUK, FRANKFORT, IL (US)

(72) Inventor: MONIF M. MATOUK, FRANKFORT, IL (US)

(21) Appl. No.: 15/091,953

(22) Filed: Apr. 6, 2016

Related U.S. Application Data
(63) Continuation of application No. 14/190,461, filed on Feb. 26, 2014.

(51) Int. Cl. A61K 33/38; A61M 11/00; A61K 9/08; A61K 33/34; A61K 33/30

(52) U.S. Cl. CPC A61K 33/38; A61K 33/34; A61K 33/30; A61K 9/08; A61M 11/00

(57) ABSTRACT

A wound care system for promoting wound healing and for preventing infections thereof and a method for producing the same includes an anti-microbial solution and a multi-layer wound dressing. The anti-microbial solution contains a mixture of colloidal silver, colloidal copper and colloidal zinc oxide mixed in sterile purified water. The multi-layer wound dressing is formed of a contact layer, an intermediate layer, and an outer layer. The solution is poured onto the wound dressing which is subsequently placed directly over a wound so as to provide and maintain a moist environment. In other embodiments, there are provided anti-microbial solutions and methods for producing the same, functioning as stand-alones and containing a mixture of at least two elements of colloidal silver, colloidal copper, and colloidal zinc.

### CROSS-REFERENCE TO RELATED APPLICATION

The present invention is a continuation-in-part application based on prior application Ser. No. 14/190,461 filed on Feb. 26, 2014 and entitled “Silver-Copper-Zinc Oxide Wound Care System”.

### BACKGROUND OF THE INVENTION

1. Technical Field

This invention relates generally to anti-microbial wound care dressings that are applied over various types of wounds. More particularly, it relates to a unique silver-copper-zinc oxide wound care system which is effective and efficient for promoting wound healing and for preventing infection thereof.

2. Prior Art

As is generally well-known in the art of wound care devices, silver has been recognized over the past several centuries for its high anti-microbial activity and sterilizing power as compared to other heavy metals. Thus, silver serves an important role in promoting wound healing and in preventing of infection in the wound. For example, silver lined water vessels were used by the Greeks and the Romans for purifying the water. Also, silver nitrate has been used for over 100 years ago to function as an anti-microbial treatment for burn wounds. Further, silver nitrate was replaced in the 1960's by silver sulfadiazir cream for the anti-microbial treatment for burn wounds.

With the advancements made in the modern sciences, the anti-microbial activity of silver and its mechanisms have been scientifically verified through systematic research conducted by the many scientists. Many research results have revealed that extremely small amounts of silver show sufficiently effective anti-microbial activity against bacteria.

It was believed that placing surface available silver in contact with a wound allows the silver to enter the wound and interact with the surface of the microbe by disrupting its surface membranes. The silver then enters into the cell to denature and to disrupt its cellular proteins so as to block its cellular respiration and to disrupt its DNA causing the cell to die thereby preventing infection of the wound and facilitating the healing process. As a result, silver-containing microbiocides have been incorporated into wound care devices and are gaining wide acceptance in the medical industry as an effective and safe way of controlling microbial growth.

In view of the foregoing background, there have been various attempts made in the prior art heretofore to the development of different types of wound care devices. Currently, wound care devices are being used in the form of gels, pastes, and various other solid-form dressings, such as sheets and foams of various sizes and shapes. However, these traditional dressings suffer from the disadvantages of being very expensive to produce.

Further, since the wound dressings may be typically required to be changed several times during a day this also adds to the high cost and thus made them unfeasible to use. Another disadvantage of the traditional wound dressings is that they must be applied by a trained medical professional due to their complexity. As a result, there are required frequent visits to a clinic so that the dressing can be changed. Alternatively, medical personnel, such doctors and nurses, are needed to visit the patients at their individual homes.

A prior art search in the United States Patent and Trademark Office directed to the subject matter of this application revealed the following U.S. Letters Patent Nos.:

| | |
|---|---|
| 5,872,788 | 7,118,761 |
| 5,998,692 | 7,807,661 |
| 6,333,093 | 8,263,116 |

In addition to the above issued prior art utility patents, there were also found U.S. Patent Application Publication Nos. 2012/0282321; 2011/0257617; and 2012/0330209. Further, there were found the following foreign patent documents: WO 2012/098298; GB 363,255; KR 2010021108; KR 100839088; KR 200374912; CN 101947330; and CN 102600497.

In U.S. Pat. No. 5,998,692 to Gilding, there is disclosed a wound dressing in FIG. 1 which includes a breathable film layer (a), an absorbent fabric layer (b) located on the side of the film layer remote from the wound to absorb exudates which has passed through the film layer, and a primary wound contact layer (c) formed of a silver alginate. A compression layer (d) formed of an elastic or resilient element may be disposed against the first side of the absorbent layer (b).

In U.S. Pat. No. 6,333,093 to Burrell et al., there is disclosed a multi-layer wound dressing 10 which is formed of a first wound-facing layer 12, a second absorbent core layer 14, and a third outer layer 16, as illustrated in FIG. 2. The first layer is formed of a perforated, non-adherent material such as cotton gauze. The second layer is formed of an absorbent material for absorbing moisture from the wound. The third layer serves as an adhesive layer to anchor the wound dressing 10 around the wound. An anti-microbial coating is preferably applied to at least to the first wound-facing layer to provide a localized anti-microbial effect next to the wound.

In U.S. Pat. No. 7,118,761 to Canada et al., there is disclosed in FIG. 4 a multi-layered wound care device which is formed of a three layer-wound care device 1 in relation to a wound 2. The wound care device 1 consist of a wound-facing layer 3 having a topically applied anti-microbial finish on its surface, a second layer 5 containing an odor absorbing agent and being attached to the wound-facing layer 3, and an outer layer 6 attached to the second layer 5.

In U.S. Pat. No. 8,263,116 to Kim et al., there is disclosed an anti-microbial moist wound dressing 10 as shown in FIG. 1. The wound dressing 10 is comprised of a medium layer 13 in which a silver-CMC compound 11 is dispersed. The medium layer 13 is disposed centrally in a pressure-sensitive adhesive layer 15 which is then laminated with an external protective layer 17. The silver-CMC compound 11 provides a moist environment for effective wound healing and inhibits the proliferation of harmful bacteria due to the anti-microbial and bactericide activity of the silver.

The remaining patents listed above but not specifically discussed are considered to be of general interest to show the state of the art in composite anti-microbial wound care dressings or devices which utilizes silver-based anti-microbial compounds for treating of wounds and/or methods for producing metal compositions for treatment of infectious wounds.

Accordingly, it would be desirable to provide a unique wound care system which effectively and efficiently promotes wound healing and prevents infections to the wound. It would be expedient that the wound care system includes a novel silver-copper-zinc oxide solution which provides sufficient anti-microbial activity and a multi-layer wound dressing which is capable of effectively absorbing wound exudates. Further, it would also be desirable that the multi-layer wound dressing be formed of a wound contact layer, an intermediate absorptive layer, and an external protective layer.

### BRIEF SUMMARY OF THE INVENTION

It is a general advantage of the present invention to provide a unique silver-copper-zinc oxide wound care system and a method for producing the same which is relatively simple and economical to manufacture and use, but yet overcomes the disadvantages of the prior art wound dressings. It is an advantage of the present invention to provide a unique silver-copper-zinc oxide wound care system which effectively and efficiently promotes wound healing and prevents infections to the wound. It is another advantage of the present invention to provide a unique silver-copper-zinc oxide wound care system which includes a novel silver-copper-zinc oxide solution which provides sufficient anti-microbial activity and a multi-layer wound dressing which is capable of effectively absorbing wound exudates. It is still another advantage of the present invention to provide a unique silver-copper-zinc oxide wound care system wherein the wound dressing is formed of a wound contact layer, an intermediate absorptive layer and an external protective layer

These and other objects, features and advantages of the invention are provided by a wound care system for promoting wound healing and for preventing infections thereof and a method for producing the same which includes a anti-microbial solution and a multi-layer wound dressing. The anti-microbial solution contains a mixture of colloidal silver, colloidal copper and colloidal zinc oxide mixed in sterile purified water. The multi-layer wound dressing is formed of a contact layer, an intermediate layer, and an outer layer. The solution is poured onto the wound dressing which is subsequently placed directly over a wound so as to provide and maintain a moist environment.

These and other features and advantages of the disclosed unique wound care system resides in the construction of parts and the combination thereof, the mode of operation and use, as will become more apparent from the following description, reference being made to the accompanying drawings that form a part of this specification wherein like reference characters designate corresponding parts in the several views. The embodiments and features thereof are described and illustrated in conjunction with systems, tools and methods which are meant to exemplify and to illustrate, not being limiting in scope.

The foregoing applies specifically to the disclosure of the parent application Ser. No. 14/190,461. A silver-copper-zinc oxide anti-microbial solution in FIG. 10A of the present invention added by way of this continuation-in-part application is quite similar to the anti-microbial solution shown in FIG. 1, except that the anti-microbial solution therein now functions as stand-alone solution which is used as a wound wash for debridement of a wound and for moistening the wound and/or moistening various kinds of commercially available wound care dressings or gauzes to be applied onto the wound. Alternatively, there is shown in FIG. 10B a silver-copper-zinc oxide anti-microbial solution which is substantially identical to the anti-microbial solution of FIG. 10A, except that the solution is housed in a spray bottle having a spray nozzle.

A second embodiment of a silver-zinc anti-microbial solution of FIG. 11A of the present invention also added by way of this continuation-in-part application is also quite similar to the anti-microbial solution of FIG. 1, except that the anti-microbial solution therein now functions as a stand-alone solution which is used as a wound wash for debridement of a wound and for moistening the wound and/or moistening various kinds of commercially available wound care dressings or gauzes to be applied onto the wound. Alternatively, there is shown in FIG. 11B a silver-zinc anti-microbial solution which is substantially identical to the anti-microbial solution of FIG. 11A, except that the solution is housed in a spray bottle having a spray nozzle.

A third embodiment of a silver-copper anti-microbial solution of FIG. 12A of the present invention also added by way of this continuation-in-part application is also quite similar to the anti-microbial solution of FIG. 1, except that the anti-microbial solution therein now functions as a stand-alone solution which is used as a wound wash for debridement of a wound and for moistening the wound and/or moistening various kinds of commercially available wound care dressings or gauzes to be applied onto the wound. Alternatively, there is shown in FIG. 12B a silver-copper anti-microbial solution which is substantially identical to the anti-microbial solution of FIG. 11A, except that the solution is housed in a spray bottle having a spray nozzle.

A fourth embodiment of a copper-zinc anti-microbial solution of FIG. 13A of the present invention also added by way of this continuation-in-part application is also quite similar to the anti-microbial solution of FIG. 1, except that the anti-microbial solution therein now functions as a stand-alone solution which is used as a wound wash for debridement of a wound and for moistening the wound and/or moistening various kinds of commercially available wound care dressings or gauzes to be applied onto the wound. Alternatively, there is shown in FIG. 13B a copper-zinc anti-microbial solution which is substantially identical to the anti-microbial solution of FIG. 13A, except that the solution is housed in a spray bottle having a spray nozzle.

### BRIEF DESCRIPTION OF THE SEVERAL VIEWS OF THE DRAWING

FIG. 1 is a plan view of an exemplary bottle of a unique silver-copper-zinc oxide solution for use with a multi-layer wound dressing, constructed in accordance with the principles of the present invention;

FIG. 2 is a graphical representation of a multi-layer wound dressing of a wound care system in accordance with the present invention which is applied to a wound;

FIG. 3 is a schematic cross-sectional view of the multi-layer wound dressing of FIG. 2, taken along the lines 3-3;

FIG. 4 is an exploded view of the multi-layer wound dressing of FIG. 3;

FIG. 5 is a schematic cross-sectional view, similar to FIG. 3, of a first embodiment of a multi-layer wound dressing of the present invention for providing heavy drainage from the wound;

FIG. 6 is a schematic cross-sectional view, similar to FIG. 3, of a second embodiment of a multi-layer wound dressing of the present invention for providing medium drainage from the wound;

FIG. 7 is a schematic cross-sectional view, similar to FIG. 3, of a third embodiment of a multi-layer wound dressing of the present invention for providing light drainage from the wound;

FIG. 8 shows an exemplary package for the multi-layer wound dressing of FIG. 7;

FIG. 9 shows an exemplary package for holding a number of the wound dressing of FIG. 5; and

FIG. 10A is a plan view of an exemplary bottle of a unique silver-copper-zinc oxide anti-microbial solution functioning as a stand-alone solution for use as a wound wash for debridement of a wound in accordance with the principles of the present invention;

FIG. 10B is a plan view of an exemplary spray bottle of a unique silver-copper-zinc oxide anti-microbial solution, substantially identical to FIG. 10A, functioning as a stand-alone solution for use as a wound wash for debridement of a wound;

FIG. 11A is a plan view of a second embodiment of an exemplary bottle of a unique silver-zinc anti-microbial solution functioning as a stand-alone solution for use as a wound wash for debridement of a wound in accordance with the principles of the present invention;

FIG. 11B is a plan view of an exemplary spray bottle of a unique silver-zinc anti-microbial solution, substantially identical to FIG. 11A, functioning as a stand-alone solution for use as a wound wash for debridement of a wound;

FIG. 12A is a plan view of a third embodiment of an exemplary bottle of a unique silver-copper anti-microbial solution functioning as a stand-alone solution for use as a wound wash for debridement of a wound in accordance with the principles of the present invention;

FIG. 12B is a plan view of an exemplary spray bottle of a unique silver-copper anti-microbial solution, substantially identical to FIG. 12A, functioning as a stand-alone solution for use as a wound wash for debridement of a wound;

FIG. 13A is a plan view of a fourth embodiment of an exemplary bottle of a unique copper-zinc anti-microbial solution functioning as a stand-alone solution for use as a wound wash for debridement of a wound in accordance with the principles of the present invention; and

FIG. 13B is a plan view of an exemplary spray bottle of a unique copper-zinc anti-microbial solution, substantially identical to FIG. 13A, functioning as a stand-alone solution for use as a wound wash for debridement of a wound.

### DETAILED DESCRIPTION OF THE INVENTION

Before explaining the disclosed embodiment in detail, it is to be distinctly understood at the outset that the present invention shown in the drawings and described in detail in association with a unique wound care system is not intended to serve as a limitation upon the scope or teachings thereof, but is to be considered merely for the purpose of convenience of illustration of one example of its application.

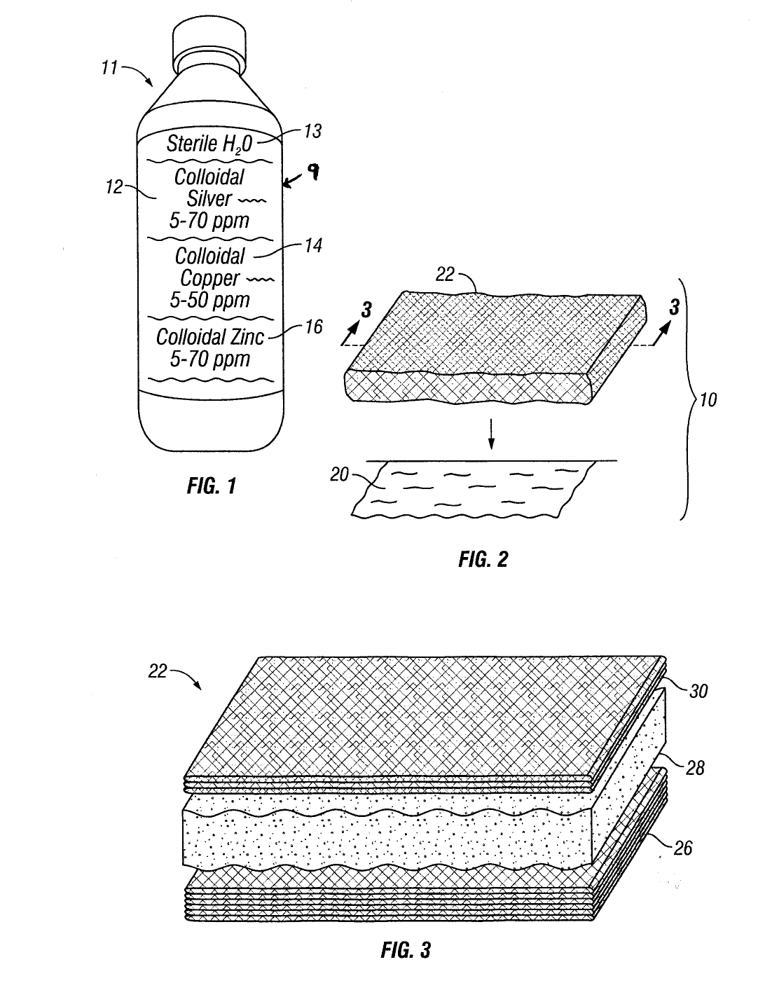

Referring now in detail to the various views of the drawings and in particular to FIGS. 1-4, there is illustrated a silver-copper-zinc oxide wound care system which is designated generally by reference numeral 10 and is constructed in accordance with the principles of the present invention. The wound care system 10 is designed to be of a unique, simplified construction, which is relatively economical to manufacture and easy to use.

With attention directed to FIG. 1, the unique solution 11 of the present invention used for cleansing of the wound and stored in a bottle 9 will now be fully described in detail. In a first step, colloidal silver 12 is added to one liter (55.6 moles) of sterile purified water (H₂O) 13. The amount of colloidal silver added to the sterile purified water is preferably in the range of 5-70 ppm. The colloidal silver is more preferably added in the amount of 20-50 ppm and is most preferably in the amount of 30 ppm, which is equivalent to 0.314700 grams/liter or 0.002917 moles/liter.

In a second step, colloidal copper 14 is further added to the mixture of the colloidal silver 12 and purified water 13. The amount of colloidal copper added to the mixture of colloidal silver 12 and sterile purified water 13 is preferably in the range of 5-50 ppm. The colloidal copper is more preferably added in the amount of 5-30 ppm and is most preferably in the amount of 10 ppm, which is equivalent to 0.089400 grams/liter or 0.00146 moles/liter.

In a third step, zinc oxide 16 is further added to the mixture of colloidal silver 12, colloidal copper 14, and purified water 13. The amount of zinc oxide added to the mixture of colloidal silver 12, colloidal copper 14, and sterile purified water 13 is preferably in the range of 5-70 ppm. The zinc oxide is more preferably added in the amount of 15-35 ppm and is most preferably in the amount of 25 ppm, which is equivalent to 0.178500 grams/liter or 0.002193 moles/liter.

As a result, after the mixing of the colloidal silver 12, colloidal copper 14, and zinc oxide 16 in the purified water 13 as just described there is obtained the unique silver-copper-zinc oxide anti-microbial solution 11 of the present invention, as illustrated in FIG. 1. The colloidal silver, colloidal copper, colloidal zinc, and sterile purified water are commercially available and can be purchased from any number of manufacturers, such as Trace Minerals Research of Roy, Utah or Purist Colloids Inc. of Westhampton, N.J. In addition, there are known compounding companies that will manufacture a solution by bottling the given chemicals with the sterile purified water, such as McGuff Pharmacy Services, Inc of Santa Ana, Calif. or US Compounding of Conway, Ark.

The solution 11 is then poured onto the wound dressing 22 of FIG. 2, which is subsequently placed directly over the wound 20. Finally, a dry sterile dressing (not shown) such as gauze or kerlix which is commercially available can then be used to cover or be wrapped around the wound dressings 22 so to stabilize the dressing and thus complete a wet-to-dry application.

The silver-copper-zinc oxide solution 11 provides a more thorough surface contact with the wound and also provides better anti-microbial activity due to the increased surface area coverage. In addition, the silver-copper-zinc oxide solution promotes better bacteriacidal activity due the combined effects through the use of two heavy metals, silver and copper. Further, the use of the zinc oxide enhances re-epithelialization and reduces inflammation in the wounds.

With reference to FIG. 2, there is illustrated a graphical representation of the silver-copper-zinc oxide wound care system 10 of the present invention for use in the treatment of acute wounds (e.g., burns) and/or chronic wounds 20 (e.g., decubitus ulcers and diabetic foot ulcers). The wound care system 10 in accordance with the invention includes an anti-microbial wound dressing 22 onto which is poured the silver-copper-zinc oxide solution 11 so as to form a wet contact layer.

The wet contact layer of the wound dressing is then placed directly over and applied or attached to the wound 20 so as to provide and maintain a moist environment which is effective in the treatment of the wound 20. Therefore, the wound care system 10 provides bactericidal and anti-microbial effects on the wound 20 so as to promote healing and prevent infections.

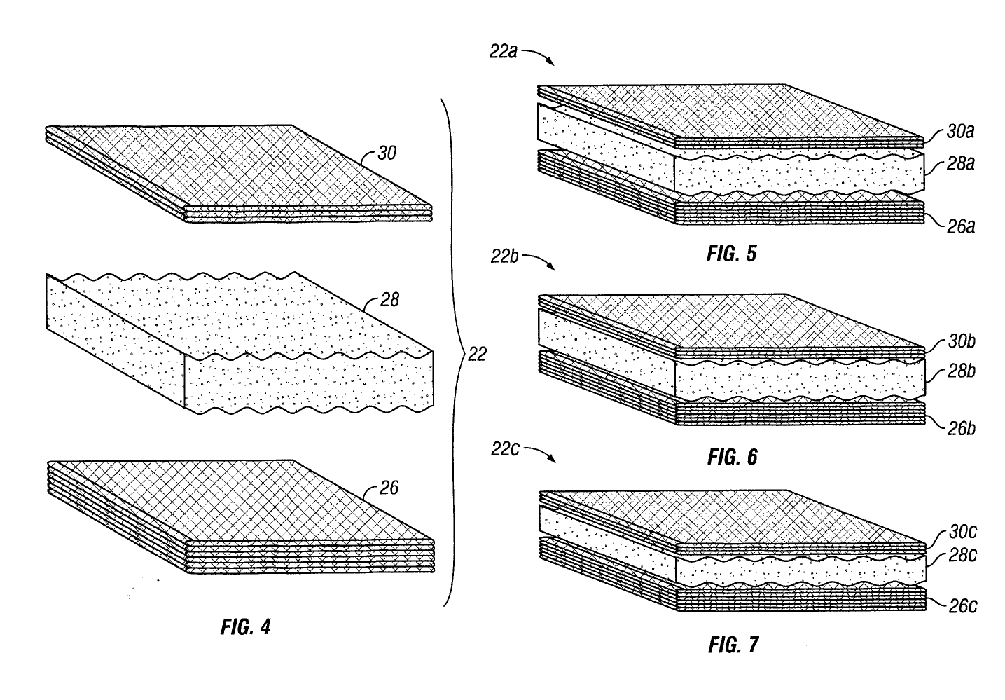

As can best be seen from FIG. 3, there is depicted a cross-sectional view of the wound dressing 22 of FIG. 1. FIG. 4 is an exploded view of the wound dressing 22 of FIG. 3. The wound dressing 22 consists of a contact layer 26, an intermediate layer 28, and outer layer 30. The contact layer 26 defining a wound-facing layer is formed of a perforated, preferably an adherent material, which adheres to the wound and permits fluids to penetrate or diffuse therethrough in a direction away from the wound 20. The perforated material may be formed of a woven or non-woven fabric.

The contact layer 26 must also be made of an absorbent material capable of effectively absorbing both the exudate secreted from the wounds and the anti-microbial metals in the solution. The contact layer 26 must also be non-occlusive so as to prevent maceration of the wound due to the presence of excessive fluids.

As illustrated in FIG. 5, there is shown a graphical representation of a first embodiment of a multi-layer wound dressing 22a. In order to provide heavy drainage from the wound, the perforated material of the contact layer 26a is preferably made of sterile, pure white, non-woven layer of pure (100%) cotton fiber or gauze having a cotton yarn in the range of 10 kw to 70 kw, or preferably 32 kw and a mesh in the range of 8 threads/cm² to 60 threads/cm², preferably 17 threads/cm².

For heavy drainage, there is used in the range of 4 to 52 layers of fiber so to provide a thickness in the range of 0.20 cm to 10.0 cm. Preferably, the number of layers of fiber is 24 so as to provide a thickness of 1.0 cm.

As illustrated in FIG. 6, there is shown a graphical representation of a second embodiment of a multi-layer wound dressing 22b. In order to provide medium drainage from the wound, the perforated material of the contact layer 26a is also preferably made of sterile, pure white, non-woven layer of pure (100%) cotton fiber or gauze having a cotton yarn in the range of 10 kw to 70 kw, or preferably 32 kw and a mesh in the range of 8 threads/cm² to 60 threads/cm², preferably 17 threads/cm².

For medium drainage, there is used in the range of 4 to 52 layers of fiber so to provide a thickness in the range of 0.20 cm to 7.0 cm. Preferably, the number of layers of fiber is 16 so as to provide a thickness of 0.7 cm.

As illustrated in FIG. 7, there is shown a graphical representation of a third embodiment of a multi-layer wound dressing 22c. In order to provide light drainage from the wound, the perforated material of the contact layer 26c is again preferably made of sterile, pure white, non-woven layer of pure (100%) cotton fiber having a cotton yarn in the range of 10 kw to 70 kw, or preferably 32 kw and a mesh in the range of 8 threads/cm² to 60 threads/cm², preferably 17 threads/cm².

For light drainage, there is used in the range of 4 to 52 layers of fiber so to provide a thickness in the range of 0.20 cm to 3.0 cm. Preferably, the number of layers of fiber is 8 so as to provide a thickness of 0.3 cm.

The wound-facing layer 26 has a width dimension of approximately four inches and a length dimension of approximately four inches. It should be clearly understood by those skilled in the art that the dimensions of the wound-facing layer can be varied in different sizes as desired so as to accommodate the different sizes of the wounds.

The intermediate layer 28 defining an absorbent layer is formed of an absorbent material for absorbing moisture from the wound or for holding moisture next to the wound in the case of a burn treatment. The absorbent material is preferably made of a natural sponge or synthetic foam which has a high moisture absorption and storage capacity so as to produce a moist environment for healing wounds effectively and for inhibiting proliferation of harmful bacteria. The intermediate layer 28 is joined to the side edges of the contact layer 26 remote from the wound-facing side.

As illustrated in FIG. 5, in order to provide for heavy drainage from the wound, the intermediate layer 28a has thickness in the range of 0.2 cm to 5.0 cm. Preferably, the thickness of the intermediate layer is 1.5 cm.

As illustrated in FIG. 6, in order to provide for medium drainage from the wound, the intermediate layer 28b has thickness in the range of 0.2 cm to 10.0 cm. Preferably, the thickness of the intermediate layer is 1.0 cm.

As illustrated in FIG. 7, in order to provide for light drainage from the wound, the intermediate layer 28c has thickness in the range of 0.2 cm to 5.0 cm. Preferably, the thickness of the intermediate layer is 0.5 cm.

The absorbent layer 28 has also a width dimension of approximately four inches and a length dimension of approximately four inches. It should be clearly understood by those skilled in the art that the dimensions of the absorption layer can likewise be varied in different sizes as desired so as to accommodate the different sizes of the wounds.

The outer layer 30 defining an external protective layer is joined to the sides edges of the intermediate layer 28 opposite the side adjacent to the contact layer 26 and acts to prevent wound exudate (secretions) absorbed in the intermediate layer 28 from being released to the outside environment, thereby maintaining a moist environment. In addition, the external protective layer serves to protect infiltration of water, bacteria, impurities and the like from the outside environment.

As illustrated in FIG. 5, in order to provide heavy drainage from the wound, the perforated material of the outer layer 30a is preferably made of sterile, pure white, non-woven layer of pure (100%) cotton fiber or gauze having a cotton yarn in the range of 10 kw to 40 kw, or preferably 32 kw and a mesh in the range of 8 threads/cm² to 60 threads/cm², preferably 29 threads/cm².

For heavy drainage, there is used in the range of 4 to 52 layers of fiber so to provide a thickness in the range of 0.20 cm to 6.0 cm. Preferably, the number of layers of fiber is 12 so as to provide a thickness of 0.6 cm.

As illustrated in FIG. 6, in order to provide medium drainage from the wound, the perforated material of the outer layer 30b is also preferably made of sterile, pure white, non-woven layer of pure (100%) cotton fiber or gauze having a cotton yarn in the range of 10 kw to 40 kw, or preferably 32 kw and a mesh in the range of 8 threads/cm² to 60 threads/cm², preferably 29 threads/cm².

For medium drainage, there is used in the range of 4 to 52 layers of fiber so to provide a thickness in the range of 0.20 cm to 4.0 cm. Preferably, the number of layers of fiber is 8 so as to provide a thickness of 0.4 cm.

As illustrated in FIG. 7, in order to provide light drainage from the wound, the perforated material of the outer layer 30c is again preferably made of sterile, pure white, non-woven layer of pure (100%) cotton fiber or gauze having a cotton yarn in the range of 10 kw to 70 kw, or preferably 32 kw and a mesh in the range of 8 threads/cm² to 60 threads/cm², preferably 29 threads/cm².

For light drainage, there is used in the range of 4 to 52 layers of fiber so to provide a thickness in the range of 0.20 cm to 2.0 cm. Preferably, the number of layers of fiber is 4 so as to provide a thickness of 0.2 cm.

The outer layer 30 has also a width dimension of approximately four inches and a length dimension of approximately four inches. It should be clearly understood by those skilled in the art that the dimensions of the outer layer can likewise be varied in different sizes as desired so as to accommodate the different sizes of the wounds.

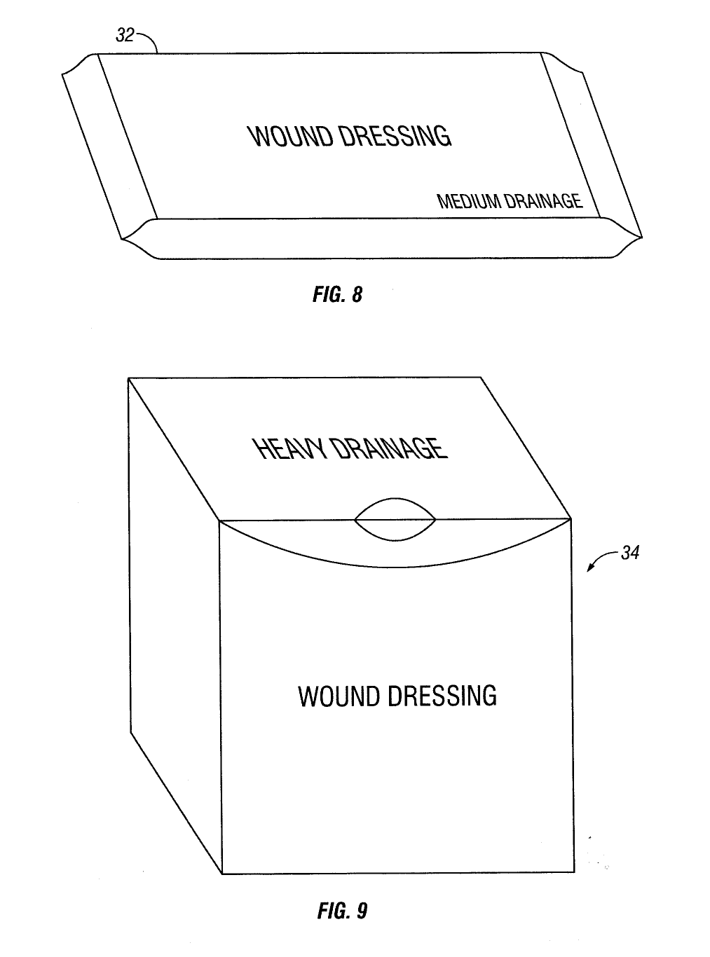

The contact layer 26, intermediate layer 28, and outer layer 30 are laminated or attached and bonded to each other by any means known in the art, such as by an adhesive, thermal pressurization or ultrasonic welding at various locations across the wound dressing 22. The wound dressing is preferably sealed in an individual package 32 as depicted in FIG. 8 and then placed in a box. Alternately, a predetermined number of wound dressings can be placed together and packaged into a larger box 34 as depicted in FIG. 9.

In use, the combination of the three different and separate vectors (colloidal silver, colloidal copper and colloidal zinc oxide) in sterile water provides increased anti-microbial activity by several folds and also reduces the potential of resistance by any bacterial strains that might be developed when any of the elements would be used alone. The wet-to-dry process created by the adherent contact 26 functioning as a wet layer and the outer layer 30 functioning as a dry layer provides a vacuum action so as to draw contaminated and excess fluids out of the wounds 20 and towards the outer layer 30. This allows for drainage to occur. As a result, there is reduced the risk of bacterial proliferation and the presence of excess fluids which may damage and break down the epithelial layer, thereby further compromising the wound.

Upon removal of the finished wound dressing, the adherent contact layer 26 serves to mechanically debride the wound by removing of loose necrotic tissue, sloughing, loosely bound tissue, and bacteria that has colonized or proliferated the wound. In addition to the removal of non-viable tissue which impedes wound closure and the reduction of bio-burden which prevents healing and re-epithelializaton, the present wound dressing mechanically aggravates the wound surface which will lead to renewed immune response, stimulation of healing and the production of granulation tissue.

In view of the above, the wound care system of the present invention will greatly reduce need for frequent clinic visits and skilled nursing home visits by medical professionals such doctors and nurses due to its simplicity of application, which can be easily learned by non-medical personnel. Therefore, this will ultimately lead to higher patient compliance rate and much better outcomes. It is believed that the elderly, underprivileged and rural population will benefit the most from this novel wound care system due to the lesser availability of health care and resources in these groups. Accordingly, this will have a significant impact on the reduction of the overall cost on medical and health care for wound care, immune-compromised and geriatric patient populations.

Further, the present wound care system has the advantage of allowing for bulk packaging of its components since the solution and the wound dressing part are stored separately, thereby reducing the costs of packaging and storage as well as prolonging its shelf life. The instant wound care system is relatively less costly when compared to currently available products on the market as it can be used in bulk by clinics, hospitals and wound care centers.

From the foregoing detailed description, it can thus been seen that the present invention provides a wound care system for promoting wound healing and for preventing infections thereof which includes a anti-microbial solution and a multi-layer wound dressing. The anti-microbial solution contains a mixture of colloidal silver, colloidal copper and zinc oxide mixed in sterile purified water. The multi-layer wound dressing is formed of a contact layer, an intermediate layer, and an outer layer. The solution is poured onto the wound dressing which is subsequently placed directly over a wound so as to provide and maintain a moist environment.

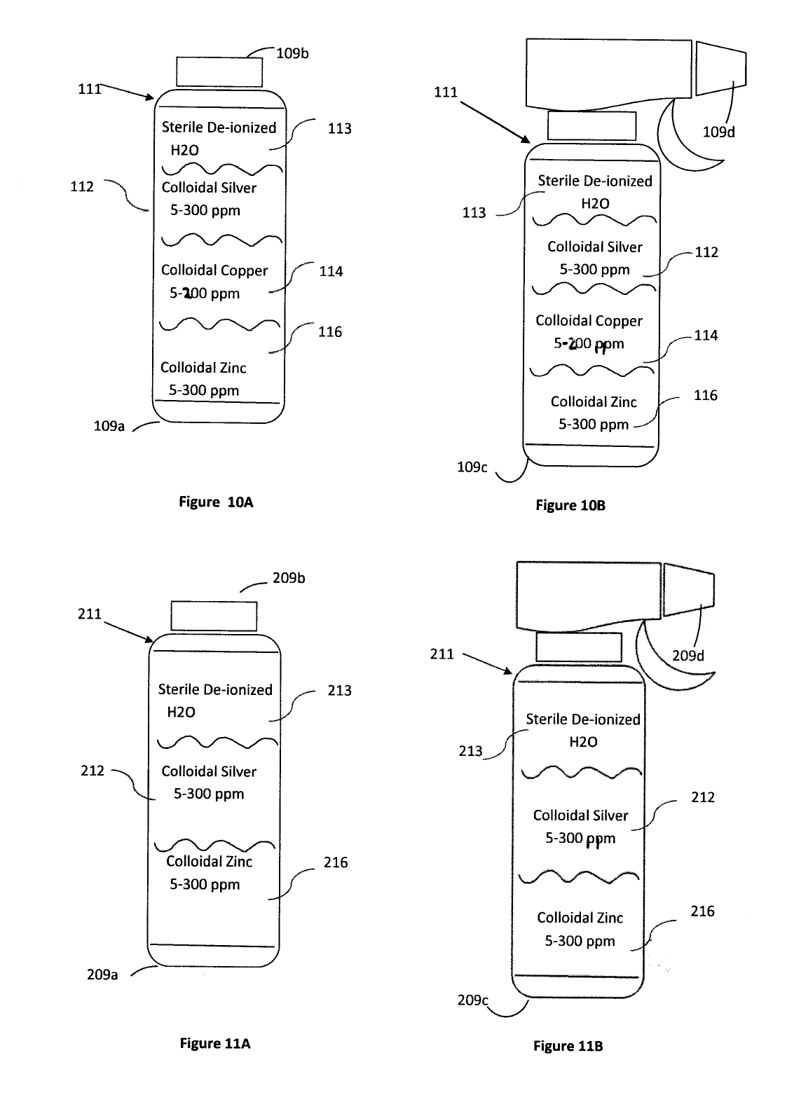

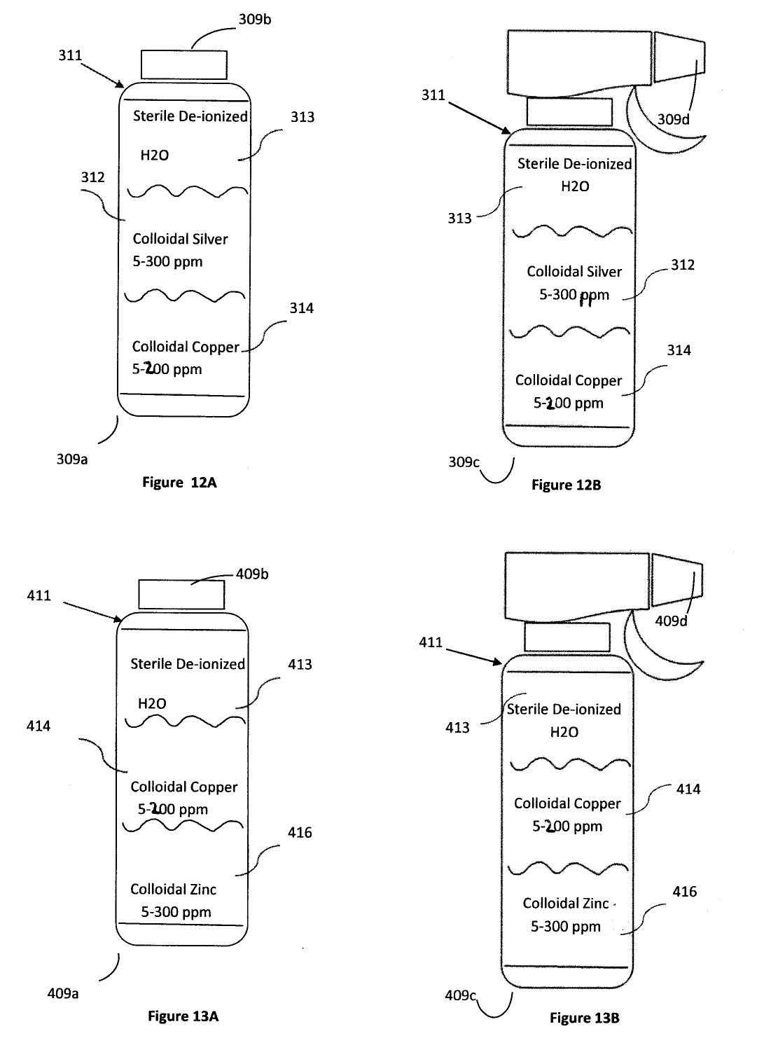

A unique anti-microbial solution containing colloidal silver, colloidal copper and colloidal zinc mixed in sterile de-ionized water is described and illustrated in connection with FIGS. 10A and 10B. A second embodiment of a unique anti-microbial solution containing colloidal silver and colloidal zinc mixed in sterile de-ionized water is described and illustrated in connection with FIGS. 11A and 11B. A third embodiment of a unique anti-microbial solution containing colloidal silver and colloidal copper mixed in sterile de-ionized water is described and illustrated in connection with FIGS. 12A and 12B. A fourth embodiment of a unique anti-microbial solution containing colloidal copper and colloidal zinc mixed in sterile ionized water is described and illustrated in connection with FIGS. 13A and 13B.

As defined herein, the phrase “colloidal silver”, “colloidal copper”, or “colloidal zinc” means nanoparticles of silver, copper or zinc suspended in sterile de-ionized water. The term “nanoparticles” of silver, copper or zinc is defined to mean clusters of atoms of silver, copper or zinc, which do not have an electrical charge.

In the unique anti-microbial solutions of the present invention, the diameter of nanoparticles of silver is preferably in the range of 0.6 to 60 nanometers (nM), more preferably less than 1 nM, and most preferably 0.6 nM. The relationship of the diameter size of the nanoparticles to its surface area is an inverse relationship. In other words, the smaller the diameter size the larger the active surface area will be so as to interact with bacteria or other substances.

Further, a colloidal solution may contain a mixture of nanoparticles and ions of the same metal. Since in solution some of the colloidal zinc may oxidize forming zinc oxide, the unique anti-microbial solutions of the present invention is referred to as being either a (1) silver-copper-zinc or silver-copper-zinc oxide solution, (2) silver-zinc or silver-zinc oxide solution, or (3) copper-zinc of copper-zinc oxide solution.

Referring now specifically to FIG. 10A of the drawings, a unique anti-microbial solution 111 of the present invention, functioning as a stand-alone solution, used as wound wash for debridement of a wound and stored in a bottle 109a with cap 109b will now be fully described in detail. In a first step, there is provided colloidal silver 112 (i.e., nanoparticles of silver suspended in sterile de-ionized water (H₂O) 113). The amount of colloidal silver is 333.33 ml and preferably in the range of 5-300 ppm. The colloidal silver is more preferably added in the amount of 15-200 ppm and is most preferably in the amount of 90 ppm, which is equivalent to 1.04523 grams/liter or 0.00969 moles/liter.

In a second step, colloidal copper 114 (i.e., nanoparticles of copper suspended in sterile de-ionized water (H₂O) 113) is further added to the colloidal silver 112. The amount of colloidal copper added to the colloidal silver 112 is 333.33 ml and is preferably in the range of 5-200 ppm. The colloidal copper is more preferably added in the amount of 15-100 ppm and is most preferably in the amount of 30 ppm, which is equivalent to 0.12105 grams/liter or 0.00191 moles/liter.

In a third step, colloidal zinc 116 (i.e., nanoparticles of zinc suspended in sterile de-ionized water (H₂O) 113) is further added to the mixture of colloidal silver 112 and colloidal copper 114. The amount of colloidal zinc added to the mixture of colloidal silver 112 and colloidal copper 114 is 333.33 ml and is preferably in the range of 5-300 ppm. The colloidal zinc is more preferably added in the amount of 15-200 ppm and is most preferably in the amount of 90 ppm, which is equivalent to 0.38472 grams/liter or 0.00588 moles/liter.

As a result, after the mixing of the colloidal silver 112, colloidal copper 114, and colloidal zinc 116 as just described there is obtained the colloidal solution 111 consisting of the elements of silver, copper and zinc with a percentage composition of nanoparticle silver being equal to 0.003%, nanoparticle copper being equal to 0.001%, nanoparticle zinc being equal to 0.003%, and sterile de-ionized water being equal to 99.993%. The concentration of the three different elements after being mixed will be as follows: (1) colloidal silver=30 ppm, (2) colloidal copper=10 ppm, and (3) colloidal zinc=30 ppm. Therefore, there will be realized one liter (55.6 moles) of the unique silver-copper-zinc anti-microbial solution 111 of the present invention, as illustrated in FIG. 10A.

With attention directed to FIG. 10B of the drawings, there is shown the unique anti-microbial solution 111 of the present invention, functioning as stand-alone solution, being used as a wound wash for debridement of a wound and stored in a spray bottle 109c having a spray nozzle 109d. Since the mixing steps to be performed are identical to the three steps just described with respect to FIG. 10A, a detailed description of the same is believed to be unnecessary and thus will not be repeated.

Referring now specifically to FIG. 11A of the drawings, a second embodiment of a unique anti-microbial solution 211 of the present invention, functioning as a stand-alone solution, used as wound wash for debridement of a wound and stored in a bottle 209a with a cap 209b will now be fully described in detail. In a first step, there is provided colloidal silver 212 (i.e., nanoparticles of silver suspended in sterile de-ionized water (H₂O) 213). The amount of colloidal silver is 500 ml and is preferably in the range of 5-300 ppm. The colloidal silver is more preferably added in the amount of 15-200 ppm and is most preferably in the amount of 60 ppm, which is equivalent to 0.69682 grams/liter or 0.00646 moles/liter.

In a second step, colloidal zinc 216 (i.e., nanoparticles of zinc suspended in sterile de-ionized water (H₂O) 213) is further added to the colloidal silver 212. The amount of colloidal zinc added to the colloidal silver 212 is 500 ml and is preferably in the range of 5-300 ppm. The colloidal zinc is more preferably added in the amount of 15-200 ppm and is most preferably in the amount of 60 ppm, which is equivalent to 0.25648 grams/liter or 0.00392 moles/liter.

As a result, after the mixing of the colloidal silver 212 and colloidal zinc 216 in the purified water 213 as just described there is obtained the colloidal solution 211 consisting of the elements of silver and zinc with a percentage composition of nanoparticle silver being equal to 0.003%, nanoparticle zinc being equal to 0.003%, and sterile de-ionized water being equal to 99.994%. The concentration of the two different elements after being mixed will be as follows: (1) colloidal silver=30 ppm and (2) colloidal zinc=30 ppm. Therefore, there will be realized one liter (55.6 moles) of the unique silver-zinc anti-microbial solution 211 of the present invention, as illustrated in FIG. 11A.

With attention directed to FIG. 11B of the drawings, there is shown the unique anti-microbial solution 211 of the present invention, functioning as stand-alone solution, being used as a wound wash for debridement of a wound and stored in a spray bottle 209c having a spray nozzle 209d. Since the mixing steps to be performed are identical to the two steps just described with respect to FIG. 11A, a detailed description of the same is believed to be unnecessary and thus will not be repeated.

Referring now specifically to FIG. 12A of the drawings, a third embodiment of a unique anti-microbial solution 311 of the present invention, functioning as a stand-alone solution, used as wound wash for debridement of a wound and stored in a bottle 309a with a cap 309b will now be fully described in detail. In a first step, there is provided colloidal silver 312 (i.e., nanoparticles of silver suspended in sterile de-ionized water (H₂O) 313). The amount of colloidal silver is 500 ml and is preferably in the range of 5-300 ppm. The colloidal silver is more preferably in the amount of 15-200 ppm and is most preferably in the amount of 60 ppm, which is equivalent to 0.69682 grams/liter or 0.00646 moles/liter.

In a second step, colloidal copper 314 is further added to the colloidal silver 312. The amount of colloidal copper added to the colloidal silver 312 is 500 ml and is preferably in the range of 5-200 ppm. The colloidal copper is more preferably added in the amount of 15-100 ppm and is most preferably in the amount of 20 ppm, which is equivalent to 0.0807 grams/liter or 0.00392 moles/liter.

As a result, after the mixing of the colloidal silver 312 and colloidal copper 314 as just described there is obtained the colloidal solution 311 consisting of the elements of silver and copper with a percentage composition of nanoparticle silver being equal to 0.003%, nanoparticle copper being equal to 0.001%, and sterile de-ionized water being equal to 99.996%. The concentration of the two different elements after being mixed will be as follows: (1) colloidal silver=30 ppm and (2) colloidal copper=10 ppm. Therefore, there will be realized one liter (55.6 moles) the unique silver-copper anti-microbial solution 311 of the present invention, as illustrated in FIG. 12A.

With attention directed to FIG. 12B of the drawings, there is shown the unique anti-microbial solution 311 of the present invention, functioning as stand-alone solution, being used as a wound wash for debridement of a wound and stored in a spray bottle 309c having a spray nozzle 309d. Since the mixing steps to be performed are identical to the two steps just described with respect to FIG. 12A, a detailed description of the same is believed to be unnecessary and thus will not be repeated.

Referring now specifically to FIG. 13A of the drawings, a fourth embodiment of a unique anti-microbial solution 411 of the present invention, functioning as a stand-alone solution, used as wound wash for debridement of a wound and stored in a bottle 409a with a cap 409b will now be fully described in detail. In a first step, there is provided colloidal copper 414 (i.e., nanoparticles of copper suspended in sterile de-ionized water (H₂O) 413). The amount of colloidal copper is 500 ml and is preferably in the range of 5-200 ppm. The colloidal copper is more preferably added in the amount of 15-100 ppm and is most preferably in the amount of 20 ppm, which is equivalent to 0.0807 grams/liter or 0.00392 moles/liter.

In a second step, colloidal zinc 416 (i.e., nanoparticles of zinc suspended in sterile de-ionized water (H₂O) 413) is further added to the colloidal silver 412. The amount of colloidal zinc added to the colloidal silver 412 is 500 ml and is preferably in the range of 5-300 ppm. The colloidal zinc is more preferably added in the amount of 15-200 ppm and is most preferably in the amount of 60 ppm, which is equivalent to 0.25648 grams/liter or 0.00392 moles/liter.

As a result, after the mixing of the colloidal copper 414 and colloidal zinc 416 as just described there is obtained the colloidal solution 411 consisting of the elements of copper and zinc with a percentage composition of nanoparticle copper being equal to 0.001%, nanoparticle zinc being equal to 0.003%, and sterile de-ionized water being equal to 99.996%. The concentration of the two different elements after being mixed will be as follows: (1) colloidal copper=10 ppm and (2) colloidal zinc=30 ppm. Therefore, there will be realized one liter (55.6 moles) of the unique copper-zinc anti-microbial solution 411 of the present invention, as illustrated in FIG. 13A.

With attention directed to FIG. 13B of the drawings, there is shown the unique anti-microbial solution 411 of the present invention, functioning as stand-alone solution, being used as a wound wash for debridement of a wound and stored in a spray bottle 409c having a spray nozzle 409d. Since the mixing steps to be performed are identical to the two steps just described with respect to FIG. 13A, a detailed description of the same is believed to be unnecessary and thus will not be repeated.

When the anti-microbial solution 111, 211, 311, or 411 illustrated in respective FIGS. 10A through 13A is being used as stand-alone solution to serve as a wound wash or as an antibacterial wound solution, or is to be used part of a system as a wetting solution for gauze or any available wound care dressing, the solution can be poured over the wound and/or over the wound dressing. The wound can then be cleansed gently by using gauze and covered with solution wetted gauze or other dressing and thereafter covered with dry gauze and wrapped, or covered with other available wound care dressing according to the instructions given for that particular dressing.

Alternatively, when the anti-microbial solution 111, 211, 311, or 411 illustrated in respective FIGS. 10B through 13B is being used as stand-alone solution to serve as a wound wash or as an antibacterial wound solution, or is to be used part of a system as a wetting solution for gauze or any available wound care dressing, the solution can sprayed directly onto the wound from the spray bottle (109c-409c) through the spray nozzle (109d-409d). The spray deposition operates by emitting the anti-microbial solution through the spray nozzle so as to atomize the solution to a certain degree. The spray bottle and spray nozzle are commercially available and can be purchased from a number of manufacturers, such as All American Containers of N. E., Inc. of Somerset, N.J. or United States Plastic Corporation of Lima. Ohio.

Suitable operating conditions include spraying the anti-microbial solution directly onto the wound through the spray nozzle which is metered and/or adjusted to provide preferably 1 to 20 ml per spray or more preferably 1 to 10 ml per spray, or most preferably 2.5 mi per spray. The pressure of the nozzle on the spray bottle is set preferably between 1 to 20 mmHg, or more preferably between 7 to 15 mmHg, or most preferably at 10 mmHg. Again, the wound can then be cleansed gently by using gauze and covered with solution wetted gauze or other dressing and thereafter covered with dry gauze and wrapped, or covered with other available wound care dressing according to the instructions given for that particular dressing.

The anti-microbial solutions in the present invention uses at two of the three nanoparticles of silver, copper and zinc in order to achieve the following outcome and thus provide the advantages over the prior art:
1) they reduce bio-burden by actively killing bacteria. All three heavy metals are bactericidal. The three elements act synergistically in this respect;
2) they reduce inflammatory response within wound environment thus reducing the presence of MMPs which destroy the wound matrix;
3) they increase formation of granular tissue and healthy wound bed by angiogenesis;
4) they remove excess drainage and contaminated fluids from the wound;
5) they deoderanize the wound;
6) they reduce scar tissue formation;
7) they reduce pain;
8) they increase epidermal tissue proliferation;
9) they increase tensile strength of newly formed tissue by increasing presence of cross linked collagen; and
10) they promote wound contraction, epithelialization and closure.

Furthermore, it is important to be able to have the ability to use different combinations of nanoparticles of silver, copper and zinc in a colloidal solution particularly in the presence of allergy or intolerance to any of the three elements.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the appended claims.

1. A anti-microbial solution for promoting wound healing and for preventing infections thereof, comprising:
providing colloidal silver in the amount of 333.33 ml and in the range of 5-300 ppm;
colloidal copper in the amount of 333.33 ml and in the range of 5-200 ppm being mixed with said colloidal silver; and
colloidal zinc in the amount of 333.33 ml and in the range of 5-300 ppm being mixed with the mixture of said colloidal silver and colloidal copper.

2. A anti-microbial solution for promoting wound healing as claimed in claim 1, wherein the amount of colloidal silver being added is preferably 90 ppm.

3. A anti-microbial solution for promoting wound healing as claimed in claim 2, wherein the amount of colloidal copper being added is preferably 30 ppm.

4. A anti-microbial solution for promoting wound healing as claimed in claim 3, wherein the amount of colloidal zinc being added is preferably 90 ppm.

5. A anti-microbial solution for promoting wound healing as claimed in claim 4, wherein said solution has a percentage of composition of nanoparticle silver=0.003%, nanoparticle copper=0.001%, nanoparticle zinc=0.003%, and sterile de-ionized water=99.993% and wherein the concentration after mixing will be colloidal silver=30 ppm, colloidal copper=10 ppm and colloidal zinc=30 ppm so as to realize one liter of the solution.

6. A anti-microbial solution for promoting wound healing as claimed in claim 1, wherein said solution is preferably housed in a plastic bottle having a cap.

7. A anti-microbial solution for promoting wound healing as claimed in claim 1, wherein said solution is preferably housed in a spray bottle having a spray nozzle.

8. A anti-microbial solution for promoting wound healing as claimed in claim 7, wherein said spray nozzle is adjusted to spray in the amount of 1 to 20 ppm per spray.

9. A anti-microbial solution for promoting wound healing as claimed in claim 8, wherein the amount of spray is preferably in the amount of 2.5 ml per spray.

10. A anti-microbial solution for promoting wound healing as claimed in claim 8, wherein said spray nozzle has its pressure set between 1-20 mmHg.

11. A method for producing a anti-microbial solution for promoting wound healing and for preventing infections thereof, said comprising the steps of:
providing colloidal silver in the amount of 333.33 ml and in the range of 5-300 ppm;
mixing of colloidal copper in the amount of 333.33 and in the range of 5-200 ppm with said colloidal silver; and
mixing of colloidal zinc in the amount of 333.33 ml and in the range of 5-300 ppm with the mixture of said colloidal silver and colloidal copper.

12. A method for producing a anti-microbial solution for promoting wound healing as claimed in claim 11, wherein the amount of colloidal silver is preferably 90 ppm.

13. A method for producing a anti-microbial solution for promoting wound healing as claimed in claim 12, wherein the amount of colloidal copper is preferably 30 ppm.

14. A method for producing a anti-microbial solution for promoting wound healing as claimed in claim 13, wherein the amount of colloidal zinc is preferably 90 ppm.

15. A method for producing a anti-microbial solution for promoting wound healing as claimed in claim 14, wherein said solution has a percentage of composition of nanoparticle silver=0.003%, nanoparticle copper=0.001%, nanoparticle zinc=0.003%, and sterile de-ionized water=99.993% and wherein the concentration after mixing will be colloidal silver=30 ppm, colloidal copper=10 ppm and colloidal zinc=30 ppm so as to realize one liter of the solution.

16. A anti-microbial solution for promoting wound healing and for preventing infections thereof, comprising:
providing colloidal silver in the amount of 500 ml and in the range of 5-300 ppm; and
one of colloidal copper in the amount of 500 ml and in the range of 5-200 ppm and colloidal zinc in the amount of 500 ml and in the range of 5-300 ppm being mixed with said colloidal silver.

17. A anti-microbial solution for promoting wound healing as claimed in claim 16, said one of colloidal copper and colloidal zinc being mixed is colloidal zinc.

18. A anti-microbial solution for promoting wound healing as claimed in claim 17, wherein the amount of colloidal silver added is preferably 90 ppm.

19. A anti-microbial solution for promoting wound healing as claimed in claim 18, wherein the amount of colloidal zinc added is preferably 90 ppm.

20. A anti-microbial solution for promoting wound healing as claimed in claim 16, said one of colloidal copper and colloidal zinc being mixed is colloidal copper.

21. A anti-microbial solution for promoting wound healing as claimed in claim 20, wherein the amount of colloidal silver is preferably 90 ppm.

22. A anti-microbial solution for promoting wound healing as claimed in claim 21, wherein the amount of colloidal copper added is preferably 30 ppm.

23. A method for producing a anti-microbial solution for promoting wound healing and for preventing infections thereof, comprising the steps of:
providing of colloidal silver in the amount of 500 ml and in the range of 5-300 ppm; and
mixing of one of colloidal copper in the amount of 500 ml and in the range of 5-200 ppm and colloidal zinc in the amount of 500 ml and in the range of 5-300 ppm with said colloidal silver.

24. A method for producing a anti-microbial solution for promoting wound healing as claimed in claim 23, wherein said one of colloidal copper and colloidal zinc being mixed is colloidal zinc.

25. A method for producing a anti-microbial solution for promoting wound healing as claimed in claim 24, wherein the amount of colloidal silver is preferably 90 ppm.

26. A method for producing a anti-microbial solution for promoting wound healing as claimed in claim 25, wherein the amount of colloidal zinc is preferably 90 ppm.

27. A method for producing a anti-microbial solution for promoting wound healing as claimed in claim 23, wherein said one of colloidal copper and colloidal zinc being mixed is colloidal copper.

28. A method for producing a anti-microbial solution for promoting wound healing as claimed in claim 27, wherein the amount of colloidal silver is preferably 90 ppm.

29. A method for producing a anti-microbial solution for promoting wound healing as claimed in claim 28, wherein the amount of colloidal copper is preferably 30 ppm.

30. A anti-microbial solution for promoting wound healing and for preventing infections thereof, comprising:
providing colloidal copper in the amount of 500 ml and in the range of 5-200 ppm; and
colloidal zinc in the amount of 500 ml and in the range of 5-300 ppm being mixed with said colloidal copper.

31. A anti-microbial solution for promoting wound healing as claimed in claim 30, wherein the amount of colloidal copper is preferably 30 ppm.

32. A anti-microbial solution for promoting wound healing as claimed in claim 31, wherein the amount of colloidal zinc is preferably 90 ppm.

33. A method for producing a anti-microbial solution for promoting wound healing and for preventing infections thereof, comprising the steps of:
providing of colloidal copper in the amount of 500 ml and in the range of 5-200 ppm; and
mixing of colloidal zinc in the amount of 500 ml and in the range of 5-300 ppm with said colloidal copper.

34. A method for producing a anti-microbial solution for promoting wound healing as claimed in claim 33, wherein the amount of colloidal copper is preferably 30 ppm.

35. A method for producing a anti-microbial solution for promoting wound healing as claimed in claim 34, wherein the amount of colloidal zinc is preferably 90 ppm.
